(19) United States
(12) Patent Application Publication
(10) Pub. No.: US 2025/0064649 A1
(43) Pub. Date: Feb. 27, 2025

(54) Intelligent Monitoring Diaper with Enhanced Features

(71) Applicant: Wu Guohui, Kowloon (CN)

(72) Inventor: Wu Guohui, Kowloon (CN)

(21) Appl. No.: 18/455,603

(22) Filed: Aug. 24, 2023

(51) Int. Cl. A61F 13/49; A61F 13/84

(52) U.S. Cl. CPC A61F 13/49003; A61F 13/84; A61F 2013/8479; A61F 2013/8488

(57) ABSTRACT

An intelligent monitoring diaper with enhanced features having: a diaper body; a monitor equipped with a battery, a main control board, a body temperature sensor, a humidity sensor, an odor sensor, a motion sensor, and a warning device; the battery electrically connected to the main control board; d. the body temperature sensor, humidity sensor, odor sensor, motion sensor, and warning module individually electrically connected to the main control board; the humidity sensor electrically connected to a conductive pogo pin; the diaper body having a conductive cable; and the monitor is detachably installed on the diaper body, with the conductive pogo pin in contact with the conductive cable.

### CROSS-REFERENCE TO RELATED APPLICATIONS

This application claims the benefit under 35 U.S.C. § 119(a) of Chinese Patent Application Ser. No. 2023216251165, filed on Jun. 6, 2023, the contents of which are incorporated herein by reference in their entirety.

### FIELD OF THE INVENTION

The present invention relates to the field of diapers and, more specifically, to an intelligent monitoring diaper with advanced capabilities.

### BACKGROUND

Conventional diapers lack proactive monitoring and timely alerts, relying on visual inspection by caregivers to assess the need for a diaper change. This method often results in delayed action and discomfort for the user.

Therefore, there is a need for an intelligent monitoring diaper with enhanced features, overcoming the limitations of the prior art.

### BRIEF DESCRIPTION OF THE DRAWINGS

These and other features, aspects and advantages of the present invention will become better understood with regard to the following description, appended claims, and accompanying figures where:

FIG. 1 is a schematic diagram of the smart monitoring diaper system;

FIG. 2: Exploded view schematic diagram of the smart monitoring diaper system; and

FIG. 3: Functional block diagram illustrating the monitor circuit.

### SUMMARY

The present invention overcomes the limitations of the prior art by providing an intelligent monitoring diaper with enhanced features. The intelligent monitoring diaper described herein comprises a diaper body and a monitor. The monitor is equipped with a battery, a main control board, a body temperature sensor, a humidity sensor, an odor sensor, a motion sensor, and a warning device. The battery is electrically connected to the main control board, while the body temperature sensor, humidity sensor, odor sensor, motion sensor, and warning module are individually connected to the main control board. The humidity sensor is linked to a conductive pogo pin, and the diaper body is equipped with a conductive cable. The monitor is detachably installed on the diaper body, with the conductive pogo pin making contact with the conductive cable.

### DETAILED DESCRIPTION OF THE INVENTION

The present invention overcomes the limitations of the prior art by providing an intelligent monitoring diaper with enhanced features.

All dimensions specified in this disclosure are by way of example only and are not intended to be limiting. Further, the proportions shown in these Figures are not necessarily to scale. As will be understood by those with skill in the art with reference to this disclosure, the actual dimensions and proportions of any system, any device or part of a system or device disclosed in this disclosure will be determined by its intended use.

Methods and devices that implement the embodiments of the various features of the invention will now be described with reference to the drawings. The drawings and the associated descriptions are provided to illustrate embodiments of the invention and not to limit the scope of the invention. Reference in the specification to “one embodiment” or “an embodiment” is intended to indicate that a particular feature, structure, or characteristic described in connection with the embodiment is included in at least an embodiment of the invention. The appearances of the phrase “in one embodiment” or “an embodiment” in various places in the specification are not necessarily all referring to the same embodiment.

Throughout the drawings, reference numbers are re-used to indicate correspondence between referenced elements. In addition, the first digit of each reference number indicates the figure where the element first appears.

As used in this disclosure, except where the context requires otherwise, the term “comprise” and variations of the term, such as “comprising”, “comprises” and “comprised” are not intended to exclude other additives, components, integers or steps.

In the following description, specific details are given to provide a thorough understanding of the embodiments. However, it will be understood by one of ordinary skill in the art that the embodiments may be practiced without these specific details. Well-known circuits, structures and techniques may not be shown in detail in order not to obscure the embodiments. For example, circuits may be shown in block diagrams in order not to obscure the embodiments in unnecessary detail.

Also, it is noted that the embodiments may be described as a process that is depicted as a flowchart, a flow diagram, a structure diagram, or a block diagram. The flowcharts and block diagrams in the figures can illustrate the architecture, functionality, and operation of possible implementations of systems, methods and computer programs according to various embodiments disclosed. In this regard, each block in the flowchart or block diagrams can represent a module, segment, or portion of code, that can comprise one or more executable instructions for implementing the specified logical function(s). It should also be noted that, in some alternative implementations, the functions noted in the blocks may occur out of the order noted in the figures. Although a flowchart may describe the operations as a sequential process, many of the operations can be performed in parallel or concurrently. In addition, the order of the operations may be rearranged. A process is terminated when its operations are completed. A process may correspond to a method, a function, a procedure, a subroutine, a subprogram, etc. When a process corresponds to a function, its termination corresponds to a return of the function to the calling function or the main function. Additionally, each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

Moreover, a storage may represent one or more devices for storing data, including read-only memory (ROM), random access memory (RAM), magnetic disk storage mediums, optical storage mediums, flash memory devices and/or other non-transitory machine readable mediums for storing information. The term “machine readable medium” includes, but is not limited to portable or fixed storage devices, optical storage devices, wireless channels and various other non-transitory mediums capable of storing, comprising, containing, executing or carrying instruction(s) and/or data.

Furthermore, embodiments may be implemented by hardware, software, firmware, middleware, microcode, or a combination thereof. When implemented in software, firmware, middleware or microcode, the program code or code segments to perform the necessary tasks may be stored in a machine-readable medium such as a storage medium or other storage(s). One or more than one processor may perform the necessary tasks in series, distributed, concurrently or in parallel. A code segment may represent a procedure, a function, a subprogram, a program, a routine, a subroutine, a module, a software package, a class, or a combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted through a suitable means including memory sharing, message passing, token passing, network transmission, etc. and are also referred to as an interface, where the interface is the point of interaction with software, or computer hardware, or with peripheral devices.

In the following description, certain terminology is used to describe certain features of one or more embodiments of the invention.

Various embodiments provide an intelligent Monitoring Diaper with Enhanced Features. One embodiment of the present invention provides an intelligent monitoring diaper with enhanced features. In another embodiment, there is provided a method for using the system. The system and method will now be disclosed in detail.

The intelligent monitoring diaper is designed to provide enhanced functionality and convenience to both users and caregivers. It includes the following features:
1. Battery-Powered Monitor: The monitor incorporates a battery, ensuring reliable and independent operation. The battery is electrically connected to the main control board, powering all essential components.
2. Comprehensive Sensor Integration: The main control board facilitates individual electrical connections with various sensors, including the body temperature sensor, humidity sensor, odor sensor, motion sensor, and warning module. These sensors enable real-time monitoring of critical parameters related to body temperature, urine, feces, and physical activity.
3. Warning Device: The monitor is equipped with a warning device that actively alerts caregivers through audible alarms and visual indicators. This feature ensures timely notifications, prompting caregivers to take necessary actions promptly.
4. Detachable Installation: The monitor is detachably installed on the diaper body, providing flexibility and convenience. The conductive pogo pin establishes contact with the conductive cable when properly installed, allowing seamless data transmission between the monitor and the diaper.

### Improvements:

1. The warning device may include a loudspeaker, an LED light, or a combination of both.
2. The monitoring device is equipped with control buttons, electrically connected to the main control board.
3. The monitoring device also features a communication module, enabling communication with smart devices.
4. The monitoring device includes a GPS locator, electrically connected to the main control board.
5. An enzyme detection module is integrated into the monitoring device, with electrical connections to both the main control board and the conductive pogo pin. Additionally, an enzyme test piece is provided on the diaper body, electrically connected to the conductive cable.
6. The diaper body comprises a waterproof layer, a water-absorbing layer, and a conductive layer. The conductive cable is positioned within the conductive layer.
7. The conductive layer contains conductive flat wires arranged in a grid-like pattern of intersecting vertical and horizontal stripes.
8. An elastic clip is situated on the back of the monitoring device, allowing for detachable clamping onto the diaper. This ensures close contact between the conductive pogo pin and the conductive cable.

### Advantages:

The intelligent monitoring diaper offers several benefits. Equipped with a monitoring device powered by a battery, it can monitor the user's body temperature, urine presence (via the humidity sensor and conductive cable), feces presence (through the odor sensor), and user activity (via the motion sensor). Upon detecting pre-set conditions, the warning device actively alerts caregivers, facilitating timely checks. The detachable installation of the monitor onto the diaper, facilitated by the conductive pogo pin and conductive cable, allows for repeated disassembly and reduces costs while promoting environmental sustainability.

The intelligent monitoring diaper significantly improves the effectiveness and efficiency of diaper usage. By incorporating advanced sensors and a warning system, caregivers are promptly notified of critical conditions such as abnormal body temperature, soiled diapers, and exercise patterns. The detachable design facilitates reusability and reduces costs effectively.

In conclusion, the intelligent monitoring diaper described herein addresses the limitations of conventional diapers, enabling proactive monitoring and timely alerts. Its innovative design and enhanced functionality provide valuable benefits to both users and caregivers, improving overall comfort and well-being.

In the following detailed description, reference is made to the accompanying drawings, which illustrate specific embodiments of the smart monitoring diaper system.

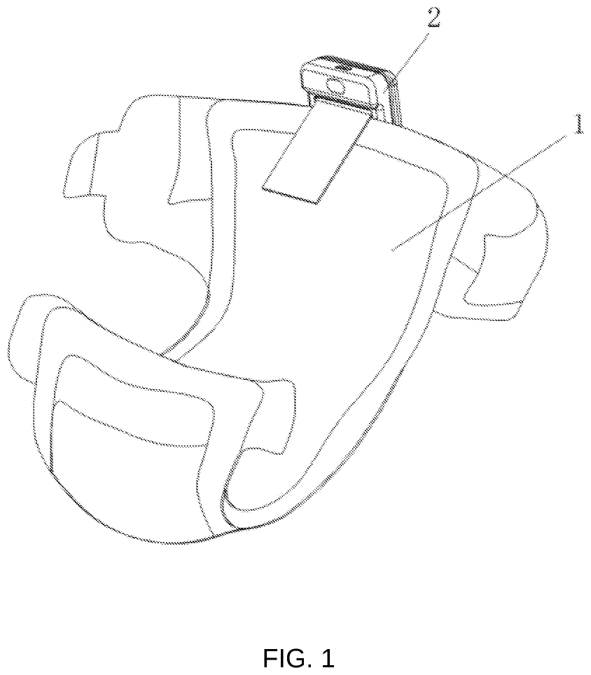

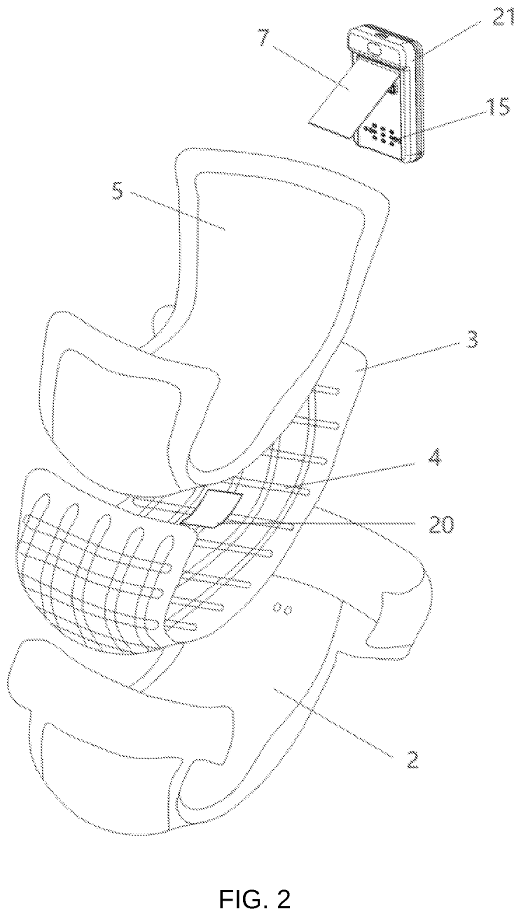

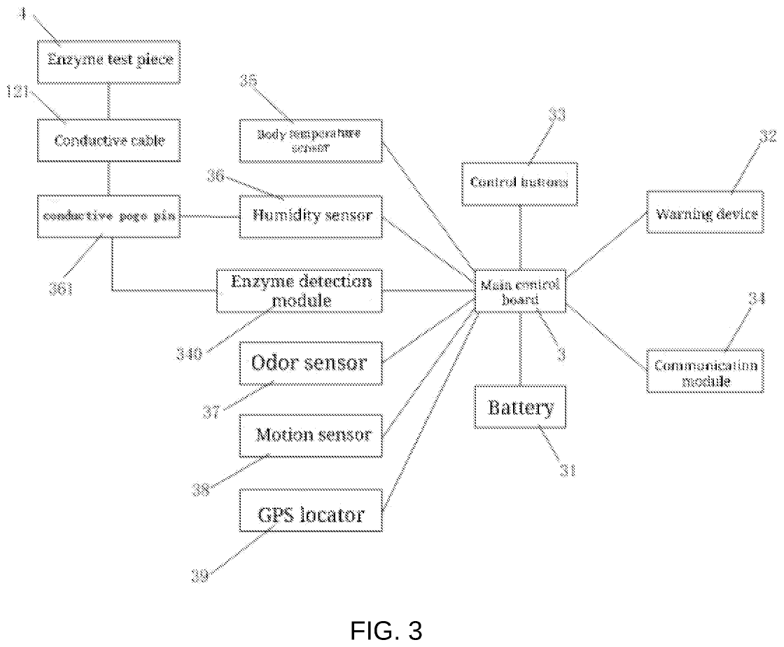

As depicted in FIGS. 1, 2 and 3, the smart monitoring diaper system comprises a diaper body (1) and a monitor (2). The monitor (2) is secured to the diaper body (1) using an elastic clip (21), allowing for easy detachment and reinstallation on different diaper bodies (1). This design ensures reusability, simple assembly, disassembly, and cost-effectiveness.

The monitor (2) includes a main control board (3), a battery (31), a warning device (32), control buttons (33), a communication module (34), a body temperature sensor (35), a humidity sensor (36), an odor sensor (37), a motion sensor (38), a GPS locator (39), and an enzyme detection module (340).

The battery (31) is electrically connected to the main control board (3) and provides power to the main control board (3), sensors, and other electronic components.

The main control board (3) is connected to the warning device (32), control buttons (33), communication module (34), body temperature sensor (35), humidity sensor (36), odor sensor (37), motion sensor (38), GPS locator (39), and enzyme detection module (340). It receives detection signals from each sensor, triggers the warning device (32) accordingly, and manages the communication between the communication module (34) and smart devices.

The warning device (32) utilizes a loudspeaker and LED light to provide timely alerts to guardians through sound and flashing lights.

Control buttons (33) enable control of the monitor's (2) power switch, warning device (32) activation, and communication module (34) connection.

The communication module (34) can employ a Bluetooth or WIFI module, enabling close-range or long-distance communication with smart devices. When the monitor (2) detects an abnormal state, it transmits abnormal information, body temperature readings, and blood sugar readings to the smart device via the communication module (34). The smart device can be a smartphone or a tablet computer.

The body temperature sensor (35) monitors the user's body temperature. If an abnormal body temperature is detected, such as fever, the warning device (32) sends a warning signal to alert the guardian. Simultaneously, the abnormal body temperature information and readings are transmitted to the smart device.

The humidity sensor (36) is connected to a conductive pogo pin (361) located on the back of the monitor (2). The diaper body (1) consists of a waterproof layer (11), a conductive layer (12), and a water-absorbing layer (13). The conductive layer (12) contains conductive cables (121) arranged in a grid pattern. When the monitor (2) is attached to the diaper body (1), a hole in the waterproof layer (11) aligns with the conductive pogo pin (361), exposing the conductive cable (121) and allowing the conductive pogo pin (361) to make contact, forming a circuit. Urine is detected through the conductive cable (121). When urine is present on the diaper body (1), it permeates the conductive cable (121), altering its resistance. Upon detecting the resistance change, the main control board (3) determines the presence of urine and triggers the warning device (32) while relaying the urine information to the smart device via the communication module (34). This enables accurate and timely awareness of urine in the diaper, facilitating prompt diaper changes to prevent discomfort or skin irritation.

The odor sensor (37) detects the presence of feces in the diaper using sensors such as H2S, NH3, C8H7N, C9H9N, CH3COOH, C4H8O2, H2, or CH4 sensors. When feces are detected, the warning device (32) issues an alert, notifying the guardian. Additionally, the feces information is transmitted to the smart device via the communication module (34).

The motion sensor (38) utilizes a three-axis or six-axis acceleration sensor to monitor the user's motion state, particularly their sleeping posture. In case of any issues, the device (32) sends a warning signal to alert the guardian and transmits the sleeping posture information to the smart device through the communication module (34). This allows the guardian to make timely adjustments and prevent suffocation caused by improper sleeping positions.

The GPS locator (39) tracks the user's walking activity and location information, which is transmitted to the smart device via the communication module (34). This enables real-time knowledge of the user's position, preventing them from getting lost.

The enzyme detection module (340) is connected to the conductive pogo pin (361). An enzyme test piece (4) is positioned on the water-absorbing layer (13) of the diaper body (1) and electrically connected to the conductive cable (121). The enzyme test piece (4) may be a uric acid test piece for measuring uric acid content. When urine is present on the diaper body (1), it infiltrates the enzyme test piece (4), causing a change in its resistance. The current is then transmitted through the conductive cable (121) to the enzyme detection module (340), which analyzes the resistance value to determine the uric acid content. The reading is subsequently sent to the smart device via the communication module (34). Different enzyme test strips (4) can be used, depending on specific requirements, such as total cholesterol test strips or high-density lipoprotein cholesterol test strips.

The aforementioned embodiments serve as illustrations of the smart monitoring diaper system and should not limit the scope of the invention. Those skilled in the art may make equivalent embodiments with partial changes or modifications based on the disclosed technical content, provided that the essential technical features of the invention are maintained. Such embodiments will still fall within the scope of the invention's technical features.

What has been described is a new and improved system for an intelligent monitoring diaper with enhanced features, overcoming the limitations and disadvantages inherent in the related art.

Although the present invention has been described with a degree of particularity, it is understood that the present disclosure has been made by way of example and that other versions are possible. As various changes could be made in the above description without departing from the scope of the invention, it is intended that all matter contained in the above description or shown in the accompanying drawings shall be illustrative and not used in a limiting sense. The spirit and scope of the appended claims should not be limited to the description of the preferred versions contained in this disclosure.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means” for performing a specified function or “step” for performing a specified function should not be interpreted as a “means” or “step” clause as specified in 35 U.S.C. § 112.

What is claimed is:

1. An intelligent monitoring diaper, comprising:
a) a diaper body;
b) a monitor comprising:
1) a battery;
2) a main control board connected to the battery;
3) a body temperature sensor connected to a conductive pogo pin;
4) a humidity sensor connected to a conductive pogo pin;
5) an odor sensor connected to the main control board;
6) a motion sensor connected to the main control board; and
7) a warning device connected to the main control board;
c) the diaper body comprises a conductive cable; and
d) the monitor detachably connected to the diaper body, with the conductive pogo pin in contact with the conductive cable.

2. The intelligent monitoring diaper of claim 1, further comprising control buttons electrically connected to the main control board, allowing user interaction and control of the monitor's functionality.

3. The intelligent monitoring diaper of claim 1, further comprising a communication module enabling communication with smart devices for data exchange and enhanced monitoring capabilities.

4. The intelligent monitoring diaper of claim 4, additionally incorporating a GPS locator electrically connected to the main control board, enabling real-time tracking of the user's location.

5. The intelligent monitoring diaper of claim 4, further comprising an enzyme detection module electrically connected to the main control board and the conductive pogo pin. The diaper body includes an enzyme test piece, which is electrically connected to the conductive cable, allowing analysis of specific substances within the diaper.

6. The intelligent monitoring diaper of claim 1, wherein the diaper body comprises a waterproof layer, a water-absorbing layer, and a conductive layer. The conductive cable is positioned within the conductive layer, ensuring reliable signal transmission.

7. The intelligent monitoring diaper of claim 7, wherein the conductive cables are distributed in a grid-like pattern, crossing vertically and horizontally within the conductive layer.

8. The intelligent monitoring diaper of claim 1, further comprising an elastic clip on the back of the monitor, enabling secure attachment to the diaper body. The conductive spring needle, also located on the back of the monitor, establishes close contact with the conductive cable when the elastic clip is engaged.
